(19) United States
(12) Patent Application Publication
(10) Pub. No.: US 2012/0035185 A1
(43) Pub. Date: Feb. 9, 2012

(54) Method of Using Flibanserin for Neuroprotection

(76) Inventor: Franco Borsini, Bad Waldsee (DE)

(21) Appl. No.: 13/276,970

(22) Filed: Oct. 19, 2011

Related U.S. Application Data
(63) Continuation of application No. 11/278,551, filed on Apr. 4, 2006, now abandoned.
Application No. 11/278,551 is a continuation of application No. 10/882,613, filed on Jul. 1, 2004, now abandoned.
Application No. 10/882,613 is a continuation of application No. 10/214,781, filed on Aug. 8, 2002, now abandoned.
(60) Provisional application No. 60/316,356, filed on Aug. 31, 2001.

(30) Foreign Application Priority Data
Aug. 10, 2001 (DE) DE 101 38 273.1

(51) Int. Cl. A61K 31/496; A61P 25/00

(52) U.S. Cl. 514/254.06

(57) ABSTRACT

The present invention relates to the use of 1-[2-(4-(3-trifluoromethyl-phenyl)piperazin-1-yl)ethyl]-2,3-dihydro-1H-benzimidazol-2-one, optionally in the form of its pharmaceutically acceptable acid addition salts and optionally in the form of its hydrates or solvates, for preparing a pharmaceutical composition with a neuroprotective activity.

### RELATED APPLICATIONS

This application is a Continuation of U.S. patent application Ser. No. 10/214,781 filed Aug. 8, 2002 which claims, as does the present application priority to U.S. Provisional Application Ser. No. 60/316,356, filed on Aug. 31, 2001, the disclosures of all of which are incorporated by reference in their entireties.

### FIELD OF THE INVENTION

The present invention relates to the use of 1-[2-(4-(3-trifluoromethyl-phenyl)piperazin-1-yl)ethyl]-2,3-dihydro-1H-benzimidazol-2-one, optionally in the form of the pharmaceutically acceptable acid addition salts thereof and optionally in the form of the hydrates or solvates thereof, for preparing a pharmaceutical composition having neuroprotective activity.

### BACKGROUND OF THE INVENTION

The compound 1-[2-(4-(3-trifluoromethyl-phenyl)piperazin-1-yl)ethyl]-2,3-dihydro-1H-benzimidazol-2-one (flibanserin) is known in the form of its hydrochloride from European Patent Application EP-A-526434 and has the following chemical structure:

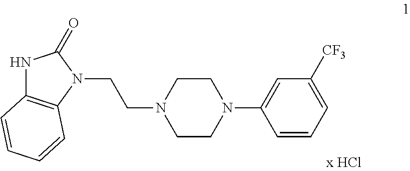

Flibanserin shows an affinity for the 5-HT_{1A} and 5-HT₂ receptor. For this reason it can be used therapeutically to treat a number of diseases. These include, for example, depression, schizophrenia, Parkinson's disease, anxiety states as well as sleep disorders, for example.

### DETAILED DESCRIPTION OF THE INVENTION

Surprisingly it has been found that the compound 1-[2-(4-(3-trifluoromethyl-phenyl)piperazin-1-yl)ethyl]-2,3-dihydro-1H-benzimidazol-2-one, optionally in the form of its pharmaceutically acceptable acid addition salts as well as optionally in the form of its hydrates or solvates, may also be used to prepare a pharmaceutical composition having a neuroprotective activity.

Accordingly, the present invention relates to the use of 1-[2-(4-(3-trifluoromethyl-phenyl)piperazin-1-yl)ethyl]-2,3-dihydro-1H-benzimidazol-2-one, optionally in the form of the pharmaceutically acceptable acid addition salts as well as optionally in the form of the hydrates or solvates, for preparing a pharmaceutical composition with a neuroprotective activity.

Preferably, the present invention relates to the use of 1-[2-(4-(3-trifluoromethyl-phenyl)piperazin-1-yl)ethyl]-2,3-dihydro-1H-benzimidazol-2-one, optionally in the form of the pharmaceutically acceptable acid addition salts as well as optionally in the form of the hydrates or solvates for preparing a pharmaceutical composition for the treatment and/or prevention of neurodegenerative diseases as well as cerebral ischaemia of various origins, selected from among epilepsy, hypoglycaemia, hypoxia, anoxia, brain trauma, brain oedema, amyotropic lateral sclerosis, Huntington's disease, Alzheimer's disease, hypotension, cardiac infarct, brain pressure (elevated intracranial pressure), ischacmic and haemorrhagic stroke (stroke), global cerebral ischaemia during stoppage of the heart, diabetic polyneuropathy, tinnitus, perinatal asphyxia, cardiac hypertrophia (thickening of the heart muscle) and cardiac insufficiency (weakness of the heart muscle).

The present invention relates, more preferably, to the use of 1-[2-(4-(3-trifluoromethyl-phenyl)piperazin-1-yl)ethyl]-2,3-dihydro-1H-benzimidazol-2-one, optionally in the form of the pharmaceutically acceptable acid addition salts as well as optionally in the form of the hydrates or solvates, for preparing a pharmaceutical composition for the treatment and/or prevention of diseases selected from among brain pressure (elevated intracranial pressure), ischaemic and haemorrhagic stroke (stroke), cardiac hypertrophia (thickening of the heart muscle) and cardiac insufficiency (weakness of the heart muscle), while particular importance is attached to the use thereof according to the invention for the treatment and/or prevention of stroke.

Optionally in the use according to the invention 1-[2-(4-(3-trifluoromethyl-phenyl)piperazin-1-yl)ethyl]-2,3-dihydro-1H-benzimidazol-2-one can be administered in combination with other active compounds. These active compounds may be selected for example from the group of glutamate receptor antagonists, calcium channel blockers, sodium channel blockers, pharmaceutically acceptable free radical scavengers, 5HT_{1A}-agonists, endothelin antagonists or proteasome inhibitors. As possible combination partners in the use of 1-[2-(4-(3-trifluoromethyl-phenyl)piperazin-1-yl)ethyl]-2,3-dihydro-1H-benzimidazol-2-one according to the invention are to be mentioned for example magnesium salts, preferably magnesium sulfate, neramexane, zonampenel, NS 1209, UK-315716, sipatrigine, crobenetine, irampanel, NS-7, harmokisane, radicut, CPI-22, DY-9760, repinotan, SUN-N4057, S-0139, citicoline or as well MLN-519. As particularly preferred combination partners in the use of 1-[2-(4-(3-trifluoromethyl-phenyl)piperazin-1-yl)ethyl]-2,3-dihydro-1H-benzimidazol-2-one according to the invention are to be mentioned magnesium sulfate, sipatrigine, crobenetine, irampanel and NS-7.

By pharmaceutically acceptable acid addition salts are meant, according to the invention, salts selected from the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid, while the salts of hydrochloric acid, hydrobromic acid, sulphuric acid, phosphoric acid and acetic acid are particularly preferred. The salts of hydrochloric acid are of particular importance.

As an alternative to being used in the form of the abovementioned pharmaceutically acceptable acid addition salts thereof the compound 1-[2-(4-(3-trifluoromethyl-phenyl)piperazin-1-yl)ethyl]-2,3-dihydro-1H-benzimidazol-2-one may also be used in the form of its free base for the purpose according to the invention. It has been found that the free base of 1-[2-(4-(3-trifluoromethyl-phenyl)piperazin-1-yl)ethyl]-2,3-dihydro-1H-benzimidazol-2-one may be obtained in two different crystal modifications, polymorphs A and B.

The formation of the different polymorphs A and B is crucially dependent on the choice of the reaction conditions used during preparation. Within the scope of the present invention, the use of polymorph A of the free base of 1-[2-(4-(3-trifluoromethyl-phenyl)piperazin-1-yl)ethyl]-2,3-dihydro-1H-benzimidazol-2-one is particularly preferred for preparing a pharmaceutical composition with a neuroprotective activity.

Polymorph A of 1-[2-(4-(3-trifluoromethyl-phenyl)piperazin-1-yl)ethyl]-2,3-dihydro-1H-benzimidazol-2-one is characterised by a melting point of about 161° C. (measured by DSC; heating rate 10 K/min). Polymorph B has a melting point of about 120° C. (measured by DSC; heating rate 10 K/min). DSC stands for Differential Scanning calorimetry.

One possible method of synthesis for preparing the free base of 1-[2-(4-(3-trifluoromethyl-phenyl)piperazin-1-yl)ethyl]-2,3-dihydro-1H-benzimidazol-2-one, particularly for preparing polymorph A, is described in the following experimental procedure:

375 kg of 1-[(3-trifluoromethyl)phenyl]-4-(2-chloroethyl)piperazine are taken up in 2500 kg of water in a suitable reactor and combined with 200 kg of aqueous NaOH solution (45% strength). 169.2 kg of 1-(2-propenyl)-1,3-dihydro-benzimidazol-2H-one, 780 kg of isopropanol, 2000 kg of water and 220 kg of aqueous NaOH solution (45% strength) are added with stirring. The reaction mixture is heated to 75-85° C. and combined first with 160 kg of concentrated hydrochloric acid then with 200 kg of water. The resulting mixture is stirred for about 45 minutes at constant temperature. After a mixture of water and isopropanol (roughly 3000 kg) has been distilled off, the residue remaining is cooled to about 65-75° C. and the pH is adjusted to about 6.5-7.5 using 125 kg of aqueous NaOH solution (45% strength). After cooling to 45-50° C. the pH is adjusted to about 8-9 by the addition of 4 kg of aqueous NaOH solution (45% strength). Then the mixture obtained is cooled to 30-35° C. and centrifuged. The residue thus obtained is washed with 340 l of water and 126 l of isopropanol. The product obtained is dried in vacuo at about 45-55° C. Yield: 358 kg of crude product;

The crude product is taken up in 1750 kg of acetone in a suitable reactor and the resulting mixture is heated to reflux temperature with stirring. The solution obtained is filtered, the filtrate is then concentrated by distillation. It is then cooled to 0-5° C. for about 1 hour, the solid that crystallises out is filtered off and finally dried at about 55° C. for about 12 hours. Yield: 280 kg of polymorph A.

Additional methods for preparing polymorph A of flibanserin are described in U.S. Publication No. US-2003-0119850-A1, which is incorporated herein by reference. Methods that may be used to prepare flibanserin may also be found in EP-526,434 A1.

Suitable pharmaceutical preparations of 1-[2-(4-(3-trifluoromethyl-phenyl)piperazin-1-yl)ethyl]-2,3-dihydro-1H-benzimidazol-2-one for use according to the invention include, for example, tablets, capsules, suppositories, solutions—particularly solutions for injection (s.c., i.v., i.m.) and infusion—syrups, emulsions or dispersible powders. The proportion of the pharmaceutically active compound in each case should be in the range from 0.1 to 90 wt. %, preferably 0.5 to 50 wt. % of the total composition, i.e. in amounts which are sufficient to achieve the dosage range specified below.

The dosage of flibanserin for use according to the invention may for example be in a range from about 0.1 to 500 mg of flibanserin per day. Preferably, the dosage is in a range from about 1-300 mg/day, more preferably in a range from about 2-200 mg/day based on flibanserin in the form of its free base. Anyone skilled in the art will see that it may possibly be necessary to depart from the quantities specified, depending on body weight or the route of administration, the individual response to the drug, the type of formulation and the time or interval at which it is administered. Thus, in some cases it may be enough to use less than the minimum amount specified, while in other cases the upper limit will have to be exceeded. When larger amounts are being administered, it may be advisable to spread them over a number of individual doses throughout the day.

Tablets containing the active substance may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, while if water is used as the diluent, for example, organic solvents may optionally be used as solubilisers or cosolvents, and the solutions are transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this to purpose, such as neutral fats or polyethyleneglycol or the derivatives thereof.

Examples of suitable excipients include for example water, pharmaceutically harmless organic solvents, such as paraffins (e.g. petroleum fractions), oils of vegetable origin (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silica and silicates), sugars (e.g. glucose, lactose and dextrose), emulsifiers (e.g. lignin, spent sulphite liquors, methylcellulose, starch and polyvinylpyrrolidone) and lubricants (e.g. magnesium stearate, talc, stearic acid and sodium laurylsulphate).

The preparations are administered in the usual way, preferably parenterally, by intravenous route, particularly by infusion. For oral use the tablets may, of course, contain, in addition to the abovementioned carriers, additives such as sodium citrate, calcium carbonate and dicalcium phosphate, together with various additives such as starch, preferably potato starch, gelatine and the like. Lubricants such as magnesium stearate, sodium laurylsulphate and talc may also be used in the tablet production. In the case of aqueous suspensions the active substances may be combined with various flavour enhancers or colourings in addition to the abovementioned excipients. For parenteral use, solutions of the active substances may be used, with suitable liquid carriers. One type of parenteral administration is by infusion, for example, which may in certain circumstances be administered over longer periods (hours or days) depending on the nature of the illness.

The following examples of formulations which can be prepared by current methods illustrate the present invention without restricting its scope:

### EXAMPLES OF PHARMACEUTICAL FORMULATIONS

| A) | Tablets | per tablet |
|---|---|---|
| | flibanserin × HCl | 10 mg |
| | lactose | 187 mg |
| | maize starch | 50 mg |
| | magnesium stearate | 3 mg |
| | | 250 mg |
| B) | Tablets | per tablet |
| | flibanserin (free base) | 80 mg |
| | lactose | 88 mg |
| | maize starch | 190 mg |
| | microcrystalline cellulose | 40 mg |
| | magnesium stearate | 2 mg |
| | | 400 mg |
| C) | Capsules | per tablet |
| | flibanserin (free base) | 10 mg |
| | lactose | 188 mg |
| | magnesium stearate | 2 mg |
| | | 200 mg |

The above mixture can be packed into suitable hard gelatine capsules.

| | |
|---|---|
| D) | Ampoule solution |

| | |
|---|---|
| flibanserin × HCl | 2 mg |
| sodium chloride | 9 mg |
| water for inj. | 5 ml |

1-4. (canceled)

5. A method for reducing neurodegeneration caused by stroke-induced ischemia during an ischaemic stroke in a warm-blooded animal comprising administering to said animal a therapeutically effective amount of 1-[2-(4-(3-trifluoromethyl-phenyl)piperazin-1-yl)ethyl]-2,3-dihydro-1H-benzimidazol-2-one or a pharmaceutically acceptable acid addition salt thereof, or a hydrate or solvate thereof, wherein the 1-[2-(4-(3-trifluoromethyl-phenyl)piperazin-1-yl)ethyl]-2,3-dihydro-1H-benzimidazol-2-one is used in the form of polymorph A of the free base.

6. The method according to claim 5, wherein 1-[2-(4-(3-trifluoromethyl-phenyl)piperazin-1-yl)ethyl]-2,3-dihydro-1H-benzimidazol-2-one is used in the form of polymorph A of the free base, having a melting point of about 161° C. as measured using Differential Scanning Calorimetry.

7. The method according to claim 5 wherein the amount administered is from about 0.1 to 500 mg per day.

8. The method according to claim 5 wherein the amount administered is from about 1-300 mg per day, based on free base form.

9. The method according to claim 5 wherein the amount administered is from about 2-200 mg per day based on the free base form.

10. The method according to claim 5 wherein the amount is administered in a dosage unit containing 80 mg of free base form.

11. The method according to claim 5 wherein the amount is administered in a dosage unit containing 10 mg of free base form.

12. The method according to claim 5 in which the amount is administered in tablet or capsule form.

13. The method according to claim 5 in which the amount is administered in solution.

14. The method of claim 5 in which 1-[2-(4-(3-trifluoromethyl-phenyl)piperazin-1-yl)ethyl]-2,3-dihydro-1H-benzimidazol-2-one or a pharmaceutically acceptable acid addition salt thereof, or a hydrate or solvate thereof is combined with an active compound comprising glutamate receptor antagonists, calcium channel blockers, sodium channel blockers, pharmaceutically acceptable free radical scavengers, 5-HT_{1A} agonists, endothelin antagonists, proteasome inhibitors, or mixtures thereof.

15. The method of claim 5 in which 1-[2-(4-(3-trifluoromethyl-phenyl)piperazin-1-yl)ethyl]-2,3-dihydro-1H-benzimidazol-2-one or a pharmaceutically acceptable acid addition salt thereof, or a hydrate or solvate thereof is combined with an active compound comprising magnesium sulfate; neramexane; zonampenel; 8-methyl-5-(4-(N,N-dimethylsulfamoyl)phenyl)-6,7,8,9-tetrahydro-1H-pyrrolo[3,2-h]-isoquinoline-2,3-dione-3-O-(4-hydroxybutyric acid-2-yl)oxime (NS-1209); (−)-6,7-dichloro-1,4-dihydro-5-[3-(methoxymethyl)-5-(3-pyridinyl)-4H-1,2,4-triazol-4-yl]-2,3-quinoxalinedione (UK-315716); sipatrigine; crobenetine; irampanel; [4-(4-fluorophenyl)-2-methyl-6-(5-piperidinopentyloxy)pyrimidine hydrochloride] (NS-7); harmokisane; radicut; disodium-[(tert-butylamino)methyl]benzene-1,3-disulfonate N-oxide (CPI-22); 3-{2-[4-(3-chloro-2-methylphenyl)-1-piperazinyl]ethyl}-5,6-dimethoxy-1-(4-imidazolylmethyl)-1H-indazole dihydrochloride 3.5 hydrate (DY-9760); repinotan; 3-chloro-4-[4-[4-(2-pyridinyl)-1,2,3,6-tetrahydropyridin-1-yl]butyl]-1,4-benzoxazepin-5(4H)-one (SUN-N4057); 27-O-3-[2-(3-carboxy-acryloylamino)-5-hydroxyphenyl]-acryloyloxy myricerone, sodium salt (S-0139); citicoline; 1R-[1S,4R,5S]-1-(1-Hydroxy-2-methylpropyl)-4-propyl-6-oxa-2-azabicyclo[3.2.0]heptane-3,7-dione (MLN-519); or mixtures thereof.

16. The method of claim 5 in which 1-[2-(4-(3-trifluoromethyl-phenyl)piperazin-1-yl)ethyl]-2,3-dihydro-1H-benzimidazol-2-one or a pharmaceutically acceptable acid addition salt thereof, or a hydrate or solvate thereof is combined with a compound comprising magnesium sulfate, sipatrigine, crobenetine, irampanel, [4-(4-fluorophenyl)-2-methyl-6-(5-piperidinopentyloxy)pyrimidine hydrochloride] (NS-7), or mixtures thereof.

17. A pharmaceutical composition comprising a therapeutically effective amount of 1-[2-(4-(3-trifluoromethyl-phenyl)piperazin-1-yl)ethyl]-2,3-dihydro-1H-benzimidazol-2-one or a pharmaceutically acceptable acid addition salt thereof, or a hydrate or solvate thereof, and an active compound comprising glutamate receptor antagonists, calcium channel blockers, sodium channel blockers, pharmaceutically acceptable free radical scavengers, 5-HT_{1A} agonists, endothelin antagonists, proteasome inhibitors, or mixtures thereof.

18. A pharmaceutical composition comprising a therapeutically effective amount of 1-[2-(4-(3-trifluoromethyl-phenyl)piperazin-1-yl)ethyl]-2,3-dihydro-1H-benzimidazol-2-one or a pharmaceutically acceptable acid addition salt thereof, or a hydrate or solvate thereof, and an active compound comprising magnesium sulfate; neramexane; zonampenel; 8-methyl-5-(4-(N,N-dimethylsulfamoyl)phenyl)-6,7,8,9-tetrahydro-1H-pyrrolo[3,2-h]-isoquinoline-2,3-dione-3-O-(4-hydroxybutyric acid-2-yl)oxime (NS-1209); (−)-6,7-dichloro-1,4-dihydro-5-[3-(methoxymethyl)-5-(3-pyridinyl)-4H-1,2,4-triazol-4-yl]-2,3-quinoxalinedione (UK-315716); sipatrigine; crobenetine; irampanel; [4-(4-fluorophenyl)-2-methyl-6-(5-piperidinopentyloxy)pyrimidine hydrochloride] (NS-7); harmokisane; radicut; disodium-[(tert-butylamino) methyl]benzene-1,3-disulfonate N-oxide (CPI-22); 3-{2-[4-(3-chloro-2-methylphenyl)-1-piperazinyl]ethyl}-5,6-dimethoxy-1-(4-imidazolylmethyl)-1H-indazole dihydrochloride 3.5 hydrate (DY-9760); repinotan; 3-chloro-4-[4-[4-(2-pyridinyl)-1,2,3,6-tetrahydropyridin-1-yl]butyl]-1,4-benzoxazepin-5(4H)-one (SUN-N4057); 27-O-3-[2-(3-carboxy-acryloylamino)-5-hydroxyphenyl]-acryloyloxy myricerone, sodium salt (S-0139); citicoline; 1R-[1S,4R,5S]-1-(1-Hydroxy-2-methylpropyl)-4-propyl-6-oxa-2-azabicyclo[3.2.0]heptane-3,7-dione (MLN-519); or mixtures thereof.

19. A pharmaceutical composition comprising a therapeutically effective amount of 1-[2-(4-(3-trifluoromethyl-phenyl)piperazin-1-yl)ethyl]-2,3-dihydro-1H-benzimidazol-2-one or a pharmaceutically acceptable acid addition salt thereof, or a hydrate or solvate thereof, a compound comprising magnesium sulfate, sipatrigine, crobenetine, irampanel, [4-(4-fluorophenyl)-2-methyl-6-(5-piperidinopentyloxy)pyrimidine hydrochloride] (NS-7), or mixtures thereof.
